(19) United States
(12) Patent Application Publication
(10) Pub. No.: US 2003/0111427 A1
(43) Pub. Date: Jun. 19, 2003

(54) Composition for controlling the dissolving rate of solid sanitizers

(75) Inventors: Ellen M. Meyer, Cleveland, TN (US); Christina D. Moss, Cleveland, TN (US)

Correspondence Address: WIGGIN & DANA LLP, ATTENTION: PATENT DOCKETING, ONE CENTURY TOWER, P.O. BOX 1832, NEW HAVEN, CT, 06508-1832, US

(73) Assignee: Arch Chemicals, Inc.

(21) Appl. No.: 10/272,186

(22) Filed: Oct. 16, 2002

Related U.S. Application Data
(60) Provisional application No. 60/334,735, filed on Oct. 19, 2001.

(51) Int. Cl. A61K 33/42; C02F 1/68

(52) U.S. Cl. 210/764; 424/602

(57) ABSTRACT

The present invention relates to a tabletted water sanitizer having improved stability and controlled solubility is disclosed and method of making the same. The tabletted sanitizer is preferably comprised of calcium hypochlorite and a dissolving rate controlling amount of poly(maleic acid) salt. Preferably, the calcium hypochlorite has at least 60% available chlorine, and most preferably at least 70% available chlorine. The tablet (or briquette) has between about 0.001% and 20% of the poly(maleic acid) salt, preferably sodium poly(maleic acid), calcium poly(maleic acid) or mixtures thereof. Tablets may be formed using any tabletting or briquetting technique commonly known in the art.

### BACKGROUND OF THE INVENTION

1. Field of the Invention

The present invention relates to novel stable tabletted compositions comprising a sanitizer and a dissolving rate controlling amount of a poly(maleic acid) salt. More particularly, the present invention relates to compositions with accurately controlled concentrations of sanitizer by reducing the solubility of the sanitizer. Reduction of the sanitizer solubility also provides for longer lasting sanitizer forms.

2. Brief Description of the Art

Calcium hypochlorite is a well-known source of available chlorine for disinfecting and sanitizing water supplies such as swimming pool water. As a source of available chlorine, calcium hypochlorite is a highly soluble material that dissolves rapidly in water. For example, at 30° C., about 21.6 grams of granular calcium hypochlorite dissolves in 100 grams of water with all of the calcium hypochlorite dissolving in less than 5 minutes. Thus, solutions of available chlorine can be provided by the direct addition of calcium hypochlorite to water. Where, however, dilute solutions are required, it is preferred to employ a dispenser that provides limited contact between solid calcium hypochlorite and the water to be treated. Even employing dispensers, however, it is somewhat difficult to supply water solutions where the concentrations of available chlorine are accurately controlled at very dilute concentrations.

Although many agents are known to modify the dissolving rate of solids, few of those agents are capable of producing stable formulations with strong oxidizers such as calcium hypochlorite that may be formed into a tablet.

For example, U.S. Pat. No. 4,087,360 to J. P. Faust et al. describes a novel composition comprising calcium hypochlorite and a proportion of a polyacrylic acid compound. The polymer reduces the solubility of the calcium hypochlorite and inhibits scale formation in a dispenser. Faust et al. do not teach the use of poly(maleic acid) in tabletted calcium hypochlorite to control dissolving rate. This patent is incorporated by reference herein in its entirety.

U.S. Pat. No. 6,146,538 to R. Martin describes a method for inhibiting scale. Although Martin discloses a ‘solid blended product’ produced by adding poly(maleic acid) salts to calcium hypochlorite during the manufacturing process, the patent does not mention further processing of the solid product into tablets, or the unexpected results of controlling the solubility of such tablets with the polymer. This patent is incorporated by reference herein in its entirety.

Accordingly, there is a need to provide a tablet with improved control of sanitizing solutions even at very dilute concentrations. There is a further need to provide formulations with a stable dissolving rate even when the formulation incorporates strong oxidizers such as calcium hypochlorite.

### BRIEF SUMMARY OF THE INVENTION

Now it has been found that improved control of sanitizing solutions can be obtained, even at very dilute concentrations. Further, it has been found that formulations with the dissolving rate modifier of this invention are stable even when those formulations incorporate strong oxidizers such as calcium hypochlorite.

Accordingly, in one embodiment of the present invention, a solid water sanitizer having improved stability and controlled solubility is disclosed. The sanitizer is selected from the group consisting of hypohalite salts, organic sanitizers, halogenated hydantoins, halogenated isocyanurates, and non-halogenated solid sanitizers and includes a proportion of a dissolving rate controlling amount of an alkali or alkali earth metal salt of poly(maleic acid). Preferably, the sanitizer is calcium hypochlorite having at least 60% available chlorine, and most preferably at least 70% available chlorine. The solid has at least about 0.001% and up to 20% sodium/calcium salt of poly(maleic acid) by weight. Most preferably, the solid is a tablet or briquette, formed using any tabletting or briquetting technique commonly known in the art.

In a second embodiment of the present invention, a method of treating water, such as a swimming pool or spa, is provided wherein a tabletted sanitizing composition is contacted with the water to be treated. The tabletted sanitizing composition is preferably comprised of calcium hypochlorite and a dissolving rate controlling amount of a poly(maleic acid) salt, preferably calcium or sodium poly(maleic acid) salt or mixtures thereof.

In a third embodiment of the present invention, a method of producing a solid water sanitizer having improved stability and controlled solubility is disclosed. In this embodiment, the solid sanitizing composition is produced by providing a sanitizing agent selected from the group consisting of hypohalite salts, organic sanitizers, halogenated hydantoins, halogenated isocyanurates, and non-halogenated solid sanitizers; precipitating poly(maleic acid) in a salt form; blending said sanitizing agent with a dissolving rate controlling amount of said poly(maleic acid) salt to form a homogenous mixture; and compacting said blended homogenous mixture into a tablet. In a fourth embodiment, the solid water sanitizer is produced using spray graining rather than blending.

### DETAILED DESCRIPTION OF THE PREFERRED EMBODIMENTS

The novel agglomerate composition of the present invention is a solid tabletted calcium hypochlorite product having a dissolving rate controlling amount of a salt of poly(maleic acid). This invention is effective for sanitizers other than calcium hypochlorite including, but not limited to, other inorganic sanitizers such as other hypohalite salts, as well as organic sanitizers such as halogenated hydantoins, halogenated isocyanurates, and non-halogenated solid sanitizers. Accordingly, the composition may include any suitable proportion of the poly(maleic acid) salt compound that is effective in reducing the solubility of the sanitizer. The upper limit of polymer concentration may be determined by the concentration of sanitizer that is required in the formulated product for sanitization activity rather than by dissolving rate characteristics.

For the purposes of this invention, a “dissolving rate controlling amount” refers to a predetermined amount of additive, in this case a salt of poly(maleic acid), to effect a desired dissolving rate of a sanitizing agent, such as calcium hypochlorite. The term “tablet” refers to granules that are compacted so as to adhere to each other by pressure and is intended to include briquettes.

Any suitable alkali or alkali earth metal salt may be employed, including, but not limited to, sodium poly(maleic acid), calcium poly(maleic acid), potassium poly(maleic acid), lithium poly(maleic acid), magnesium poly(maleic acid), and mixtures thereof. For economic reasons, however, sodium poly(maleic acid), calcium poly(maleic acid), and mixtures thereof are particularly preferred.

The poly(maleic acid) salts may be produced using any method commonly known in the art, including for example, the method disclosed in U.S. Pat. No. 6,146,538 to Martin. More particularly, the poly(maleic acid) salt may be produced by precipitating poly(maleic acid) in salt form. Such as by precipitating lime (CaO) with poly(maleic acid) in water. Alternatively, as discussed below, the poly(maleic acid) salt may be produced by mixing poly(maleic acid) with CaCl₂.2H₂O and NaOH. Any suitable molecular weight of poly(maleic acid) may be used to make these salts. The preferred molecular weight of the poly(maleic acid) is between about 500 and about 1000.

The novel composition of the present invention is in solid form and may be prepared by any of several well known methods including direct mixing or blending of the components or, by applying a coating of the poly(maleic acid) compound to particles of the hypochlorite salt, for example, by spray graining. While the composition of the present invention may be produced in any suitable form, such as granules, pellets or tablets depending on the intended use of the product, it is preferably in the form of homogenous pressed/compacted tablets.

As one skilled in the art would recognize, granular sanitizing agents, such as calcium hypochlorite, may be tabletted by introducing the granules into conventional compaction devices and compacting with pressure the granules into the shape desired, e.g., a tablet. Such devices that may be used to prepare compacted tablets include molding presses, tabletting presses, roll-type presses, pellet mills and screw extruders.

The compressed tablet or briquette useful in this invention may typically have a mass of between about 1 gram and about 350 grams or more, preferably between about 7 and 300 grams. The compressed tablet may be of a size which may be inserted readily into a skimmer or dissolving basket used with swimming pools or dissolvers used to form concentrated solutions of calcium hypochlorite. In the case of a 7 gram briquette, it is preferred that the tablet have the following dimensions: (i) between about ¾ inch and about 2 inches, preferably ¼ inches, (ii) between about ½ inch and about 1 inch, preferably ¼ inch, and (iii) between about ¼ and ¾ inch, preferably ½ inch.

The novel agglomerate composition may be used in typical calcium hypochlorite feeders, or in ‘floaters’ such as those used for feeding chlorinated isocyanurates.

The following Examples are provided to further illustrate the invention without any intention of being limited thereto. All parts and percentages are by weight unless explicitly stated otherwise.

### EXAMPLE 1

A sodium/calcium salt of poly(maleic acid) was prepared by mixing commercially available 50% aqueous solution by weight poly(maleic acid) (molecular weight 500-1000) with CaCl₂.2H₂O and 50% aqueous solution by weight NaOH in the following weight proportions: 66:6:28 respectively. The polymer salt was dried and milled to obtain particles <300 micron (50 mesh).

A homogeneous mixture containing 98.7% by weight of calcium hypochlorite [70% by weight of Ca(OCl)₂] and 1.3% by weight of sodium/calcium salt of poly(maleic acid) was prepared. The mixture was then used to form a batch of tablets having a weight of about 7 grams. The dimensions of the tablets were about 1¼ inch×¾ inch×½ inch.

The rate of solution of the tablets was measured by placing 51.94 grams of tablets into a porous basket that was suspended in 8 liters of stirring water at 30° C. At specific time intervals samples of water were removed and the chlorine concentration was measured using standard titration techniques with sodium thiosulfate. The chlorine concentration was monitored in this way until the tablets were completely dissolved. After all of the tablets were dissolved, a final sample of the water was titrated. The % rate of solution was calculated as follows:

$$\% Rate of solution = \frac{( m L thiosulfate for sample at observed time )}{m L thiosulfate for final sample} \times 100$$

The results are reported in Table 1 below.

### Comparative Test A

A portion of the calcium hypochlorite containing 70% by weight of Ca(OCl)₂ used in Example 1 was formed into tablets. The dimensions of the 7 gram tablets were about 1¼ inch×¾ inch×½ inch.

The rate of solution of the tablets was measured by placing 52.21 grams of tablets into a porous basket that was suspended in 8 liters of stirring water at 30° C. At specific time intervals samples of water were removed and the chlorine concentration was measured using standard titration techniques with sodium thiosulfate. The chlorine concentration was monitored in this way until the tablets were completely dissolved.

The results shown in Table 1 demonstrate that the dissolving rate of the tablets with poly(maleic acid) was much slower than the dissolving rate of the tablets without any polymer.

TABLE 1

| | Example 1 | | Comparative Test A | |
|---|---|---|---|---|
| Time, | Thiosulfate, | % Rate of | Thiosulfate, | % Rate of |
| hours | mL | solution | mL | solution |
| 1 | 3.69 | 38 | 6.80 | 62 |
| 2 | 6.00 | 62 | 10.01 | 92 |
| 3 | 7.50 | 77 | 10.90 | 100 |
| 4 | 8.67 | 89 | | |
| 5 | 9.50 | 98 | 10.88 | 100 |
| 6 | 9.71 | 100 | | |

### Stability Tests

The stability of the formulations described in Example 1 and Comparative Test A was tested by placing the blended powders into a series of sealed glass vials. The vials were placed in an oven at 45° C. Vials were removed at specific time intervals and the contents were tested for available chlorine. The % loss was calculated as follows:

$$\% loss = \frac{\begin{matrix}( \% available chlorine at time 0 - \\ \% available chlorine at observed time )\end{matrix}}{( \% available chlorine at time 0 )} \times 100$$

The results shown in Table 2 demonstrate that there are negligible differences between the plain calcium hypochlorite tablets and those containing the poly(maleic acid) salt.

TABLE 2

| | |
|---|---|
| Example 1 | Comparative Test A |

| Time, | Available | | Available | |
|---|---|---|---|---|
| days | chlorine, % | % Loss | chlorine, % | % Loss |
| 0 | 68.4 | | 69.3 | |
| 5 | 67.2 | 2 | 68.5 | 1 |
| 10 | 65.2 | 5 | 66.6 | 4 |
| 20 | 58.9 | 14 | 62.1 | 10 |
| 30 | 52.8 | 23 | 56.3 | 19 |

### Dissolving Rate Tests

Homogeneous mixtures containing varying amounts of calcium hypochlorite and the sodium/calcium salt of poly(maleic acid) from Example 1 were prepared. The mixtures were pressed into 20 gram tablets with 1 inch diameters. The rate of solution was measured using the technique from Example 1, except that three tablets were placed into the basket. The % rate of solution results are reported in Table 3. The results clearly demonstrate that the dissolving rate of the tablets with poly(maleic acid) was much slower than the dissolving rate of the tablets without any polymer.

TABLE 3
% Rate of Solution for Calcium Hypochlorite Tablets with Varying
Amounts of Poly(maleic acid) Salt.

| | | | | | | | |
|---|---|---|---|---|---|---|---|
| | 0% | 0% | 0.5% | 0.5% | 1.0% | 1.3% | 1.3% |
| Time | Polymer | Polymer | Polymer | Polymer | Polymer | Polymer | Polymer |

| | | | | | | | |
|---|---|---|---|---|---|---|---|
| 1 | 70 | 75 | 35 | 33 | 23 | 27 | 28 |
| 2 | 94 | 97 | 50 | 47 | 41 | 41 | 43 |
| 2.5 | 97 | 99.6 | | | | | |
| 3 | 99.9 | 100 | 64 | 61 | 52 | 59 | 56 |
| 3.5 | 100 | | | | | | |
| 4 | | | 76 | 72 | 62 | 68 | 67 |
| 5 | | | 86 | 83 | 70 | 78 | 76 |
| 6 | | | 90 | 92 | 83 | 86 | 84 |
| 7 | | | 96 | 98 | 89 | 91 | 90 |
| 8 | | | 98 | 99 | 96 | 96 | 95 |
| 9 | | | 100 | 99.9 | 98 | | |
| 9.5 | | | | 100 | 99 | | |
| 10 | | | | | 100 | 99 | 99 |
| 10.5 | | | | | | 100 | 99.9 |
| 11 | | | | | | | 100 |

While the invention has been described with reference to specific embodiments thereof, it is apparent that many changes, modifications, and variations can be made without departing from the inventive concept disclosed herein. Accordingly, it is intended to embrace all such changes, modifications and variations that fall within the spirit and broad scope of the appended claims. All patent applications, patents and other publications cited herein are incorporated by reference in their entirety.

What is claimed is:

1. A stable solid composition to sanitize water having controlled dissolution comprised of a composition having dissolving rate controlling amount of alkali salt of poly(maleic acid) and a sanitizing agent selected from the group consisting of calcium hypochlorite, hypohalite salts, organic sanitizers, halogenated hydantoins, halogenated isocyanurates, and non-halogenated solid sanitizers, wherein said composition is in the form of a pressed tablet.

2. The solid composition of claim 1 wherein said sanitizer contains at least 60% available chlorine.

3. The solid composition of claim 2 wherein said sanitizer is calcium hypochlorite.

4. The solid composition of claim 3 wherein said dissolving rate controlling amount is between about 0.001% and 20% of said alkali salt of poly(maleic acid) by weight.

5. The solid composition of claim 4 wherein said alkali salt of poly(maleic acid) is selected from the group consisting of sodium poly(maleic acid), calcium poly(maleic acid), potassium poly(maleic acid), lithium poly(maleic acid), magnesium poly(maleic acid), and mixtures thereof.

6. The solid composition of claim 5 wherein the alkali salt of poly(maleic acid) is sodium poly(maleic acid).

7. The solid composition of claim 5 wherein the alkali salt of poly(maleic acid) is calcium poly(maleic acid).

8. The solid composition of claim 5 wherein the alkali salt of poly(maleic acid) is a mixture of sodium poly(maleic acid) and calcium poly(maleic acid).

9. A method of treating water with a solid tabletted sanitizing composition comprising:
a) providing water to be treated; and
b) contacting said tabletted sanitizing composition with said water, wherein said tabletted sanitizing composition is comprised of calcium hypochlorite and a dissolving rate controlling amount of a poly(maleic acid) salt.

10. The method of claim 9 wherein said dissolving rate controlling amount of poly(maleic acid) salt is between about 0.001% and 20% by weight.

11. The method of claim 10 wherein said poly(maleic acid) salt is calcium poly(maleic acid).

12. The method of claim 10 wherein said poly(maleic acid) salt is sodium poly(maleic acid).

13. A method of preparing a solid tabletted sanitizing composition having a dissolving rate controlling amount of a poly(maleic acid) salt, comprising the steps of:
a) providing a sanitizing agent selected from the group consisting of calcium hypochlorite, hypohalite salts, organic sanitizers, halogenated hydantoins, halogenated isocyanurates, and non-halogenated solid sanitizers;
b) precipitating poly(maleic acid) in a salt form;
c) blending said sanitizing agent with a dissolving rate controlling amount of said poly(maleic acid) salt to form a homogenous mixture; and
d) compacting said blended homogenous mixture into a tablet form.

14. The method of claim 13 wherein said sanitizing agent is calcium hypochlorite.

15. The method of claim 14 wherein said dissolving rate controlling amount of poly(maleic acid) salt is between about 0.001% and 20% by weight.

16. The method of claim 15 wherein said poly(maleic acid) salt is calcium poly(maleic acid).

17. The method of claim 15 wherein said poly(maleic acid) salt is sodium poly(maleic acid).

18. A method of preparing a solid tabletted sanitizing composition having a dissolving rate controlling amount of a poly(maleic acid) salt, comprising the steps of:
a) providing a sanitizing agent selected from the group consisting of calcium hypochlorite, hypohalite salts, organic sanitizers, halogenated hydantoins, halogenated isocyanurates, and non-halogenated solid sanitizers;
b) precipitating poly(maleic acid) in a salt form;
c) spray graining said sanitizing agent with a dissolving rate controlling amount of said poly(maleic acid) salt to form a homogenous mixture; and
d) compacting said blended homogenous mixture into a tablet form.

19. The method of claim 18 wherein said sanitizing agent is calcium hypochlorite.

20. The method of claim 19 wherein said dissolving rate controlling amount of poly(maleic acid) salt is between about 0.001% and 20% by weight.

21. The method of claim 20 wherein said poly(maleic acid) salt is calcium poly(maleic acid).

22. The method of claim 20 wherein said poly(maleic acid) salt is sodium poly(maleic acid).
